(19) United States
(12) Patent Application Publication
(10) Pub. No.: US 2021/0062133 A1
(43) Pub. Date: Mar. 4, 2021

(54) Device and bioreactor monitoring system and method

(71) Applicant: Nirrin Technologies, Inc., Billerica, MA (US)

(72) Inventors: Bryan A. Hassell, Cambridge, MA (US); David P. Marchessault, Newton, MA (US); Christopher R. Saulnier, Medford, MA (US); John C. Ho, Sudbury, MA (US)

(21) Appl. No.: 17/006,172

(22) Filed: Aug. 28, 2020

Related U.S. Application Data
(60) Provisional application No. 62/892,702, filed on Aug. 28, 2019.

(51) Int. Cl. C12M 1/26; G01N 21/359; C12M 1/12; C12M 1/34

(52) U.S. Cl. CPC C12M 33/12; C12M 41/36; C12M 37/02; G01N 21/359

(57) ABSTRACT

A device for monitoring a bioreactor includes a sample tube for withdrawing a sample from a bioreactor into a sample cell and elements for analyzing the sample, in the NIR region, for example. Collecting and releasing the sample from and into the bioreactor is conducted using a peristaltic pump that is operated as a reversible/reciprocating pump. A sterile filter separates sample cell tubing from tubing connecting to the peristaltic pump.

### RELATED APPLICATIONS

This application claims the benefit under 35 USC 119(e) of U.S. Provisional Application No. 62/892,702 filed on Aug. 28, 2019, which is incorporated herein by this reference in its entirety.

### BACKGROUND OF THE INVENTION

Many processes in the chemical, biochemical, pharmaceutical, food, beverage and in other industries require some type of monitoring.

Sensors have been developed and are available to measure pH, dissolved oxygen (DO), temperature or pressure in-situ and in real-time.

Common techniques for detecting chemical constituents include high performance liquid chromatography (HPLC), gas chromatography-mass spectroscopy (GCMS), or enzyme- and reagent-based electrochemical methods. While considered accurate, many existing approaches are conducted off-line, tend to be destructive with respect to the sample, often require expensive consumables and/or take a long time to complete. In many cases, the equipment needed to perform these analyses is expensive, requires involved calibrations, and trained operators. Procedures may be time- and labor-intensive, often mitigated by decreasing the sampling frequency of a given process, thus reducing the data points. Often, samples are run in batches, after the process has been completed, yielding little or no feedback for adjusting conditions on an ongoing basis. Drawbacks such as these can persist even with automated sampling operations.

Various optical spectroscopy approaches are available to assess components, also referred to as analytes, in a sample. Among these, probably the most common is absorption spectroscopy. Incident light excites electrons of the analyte from a low energy ground state into a high energy, excited state, and the energy can be absorbed by both non-bonding n-electrons and π-electrons within a molecular orbital. Absorption spectroscopy can be performed in the ultraviolet, visible, and/or infrared region, with analytes of varying material phases and composition being interrogated by specific wavelengths or wavelength bands of light. The resulting transmitted light is then used to resolve the absorbed spectra, to determine the analyte's or sample's composition, temperature, pH and/or other intrinsic properties for applications ranging from medical diagnostics, pharmaceutical development, food and beverage quality control, to list a few.

Another option is Raman spectroscopy, which works by the detection of inelastic scattering of typically monochromatic light from a laser.

### SUMMARY OF THE INVENTION

A need exists for robust, hands-free, non-destructive, real time techniques for identifying and/or quantifying constituents in a given process. Typically, the process is conducted in a vessel, e.g., a bioreactor. The contents of the bioreactor can change as the process unfolds and data obtained by the procedures and equipment described herein can be used to monitor, adjust and/or control process parameters.

In many of its aspects, the invention relates to a device and/or method for monitoring, in-situ, an ongoing process, such as, for example, a process conducted in a bioreactor. Cells and/or substances present in the bioreactor (or another vessel) can be identified and often quantified using a suitable technique. In many implementations, the technique is near infrared (NIR) absorption spectrometry. Other optical analytical methods can be employed in the alternative or in parallel.

The device can be or can include disposable components. Typically, the device combines collection capabilities and elements needed to analyze the sample, e.g., in the NIR region of the electromagnetic spectrum. Samples can be collected from the bioreactor (or another vessel), analyzed in real time, in a nondestructive manner, and can be returned to the bioreactor once the analysis is completed. Many implementations utilize a peristaltic pump that is operated as a reversible/reciprocating pump. A sterile filter can be used to separate conduits occupied by the bioreactor sample from the pumping system.

Whereas many existing approaches rely on removing and/or circulating cells in loops external to the process vessel, typically through a pumping system, the device and procedure described herein reduce or minimize the exposure of the bioreactor sample to conditions external to the bioreactor. In addition, cells are prevented from being drawn into the pumping system.

Techniques such as the ones described herein also improve the quality of the analysis. For instance, the absence of patch fiber optics, mirrors, and so forth yields an optimized spectroscopic signal, with light traveling directly from the laser launch fiber, through the sample, into the detector. Implementations that employ round cuvettes reduce costs, while enhancing the optical signal.

Detachable parts, which can be assembled and disassembled as needed, offer flexibility and convenience. Disposable components simplify and speed up the analysis process. For example, optical elements are kept separate and can be used repeatedly, for different scans or processes, without a need for sterilization, while sampling elements are provided independently, autoclaved and/or disposed of according to a desired protocol. In addition, many of the arrangements described herein reduce the number of elements (components) that need to be sterilized.

In general, according to one aspect, the invention features a device for monitoring a bioreactor, the device comprising a sample cell, which cell includes a first end connectable for fluid communication with a sample tube for collecting a sample from a bioreactor and a second end connectable for fluid communication with a pump. The sample cell is mountable onto a tether head which includes one or more optical elements for analyzing the sample.

In embodiments, wherein the sample cell is disposable. It can further include a sterile filter at the second end. The cell might include a round cuvette and a tortuous fluid path or straight fluid path.

Typically, the pump is a peristaltic pump.

Usually, the optical elements include elements for near infrared interrogation and/or detection of analytes and might form an optical path that intersects the sample cell at a scan area.

In general, according to another aspect, the invention features a method for monitoring a bioreactor process. This method comprises operating a pump, such as a peristaltic pump, to generate a negative pressure in a sample cell, drawing medium from a bioreactor through a sample tube to collect a sample in the sample cell, analyzing the sample, and operating the pump to generate a positive pressure, thereby releasing the sample from the sample cell, through the sample tube, and into the bioreactor.

In general, according to another aspect, the invention features a device for monitoring a bioreactor in-situ, the device comprising a sample tube for extracting a sample from a bioreactor, a tether head housing one or more optical components, a peristaltic pump, a sample cell that is mountable onto or into the tether head, the sample cell having a first end that is connectable to the sample tube and a second end that is connectable to the peristaltic pump, and a sterile filter at the second end.

In general, according to another aspect, the invention features a system comprising a bioreactor, a probe that includes a sample tube immersible in the bioreactor, a sample cell having a first end configured for fluid communication with the sample tube and a second end configured for fluid communication with a pump, a sterile filter separating the sample cell from the pump, a tether head containing elements for analyzing a sample in the sample cell and configured to cover the sample cell. Finally, a controller operates the pump, analyzes the sample, or both.

In general, according to still another aspect, the invention features a device for monitoring a bioreactor. This device comprises a sample cell having a first end connectable for fluid communication with a sample tube for collecting a sample from a bioreactor, a second end connectable for fluid communication with a peristaltic pump configured for operating as a reciprocal pump, a sterile filter at the second end. Finally, system for analyzing the sample is provided.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1A and 1B are front perspective and side plan views of a sampling and analysis device, including, according to some embodiments of the invention, a tether head, an in-situ sample tube, and a disposable flow cell;

FIG. 2 is a cross-sectional view of the device of FIGS. 1A and 1B through the tether head, showing the optical elements for the sample analysis;

FIG. 3 is an exploded view of optical components housed in the tether head of the device of FIGS. 1A and 1B;

FIG. 4 is a cross-sectional view of a consumable sample cell connected to a tube that can be inserted in a bioreactor;

FIG. 5 is an exploded view of a consumable sample cell according to embodiments of the invention;

FIG. 6 is a flow diagram showing an in-situ probe operation process conducted in the collection and analysis of a bioreactor sample according to another embodiment;

FIGS. 7A and 7B are views of a sampling and analysis device including a tether head, a 45° in-situ sample tube and straight path flow cell;

FIG. 8 is a cross-sectional view of a flow cell that includes a straight pathway;

FIG. 9 is an exploded view of a flow cell that includes a straight pathway;

FIG. 10 presents an arrangement for monitoring a bioreactor using embodiments described herein;

FIG. 11 is a series of plots showing viable cell densities under various sampling and analysis conditions;

FIG. 12 provides a comparison of absorbance spectra measured in flat versus round cuvette surfaces;

FIG. 13 are plots in which samples of increasing cell densities are scanned in the NIR wavelengths, leading to increased scatter of the beam and thus an apparent increased absorbance;

FIG. 14 shows a comparison of cell density versus time, measured by cytometry and NIR spectrometry;

FIG. 15 presents a comparison of samples of Pichia growing in a shake flask. NIR measurements were performed with an in-line probe taking samples automatically and reading absorbance at approximately 1450 nanometers (nm) of wavelength and comparing to off-line measurements from a standard spectrophotometer.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In many of its aspects, the invention relates to a device and method for collecting and analyzing one or more samples during an ongoing process. Cell and/or other constituents can be detected, at various time intervals and the data can be used to assess conditions and, if necessary, adjust or optimize process parameters.

Analysis can utilize a spectroscopy system for determining the spectral response of the components in the sample cell in one or more of the following spectral regions: millimeter, microwave, terahertz, infrared (including near-, mid- and/or far-infrared), visible, ultraviolet (UV) (including vacuum ultraviolet(VUV)), x-rays and/or gamma rays. Further, the spectroscopy system can measure different characteristics, such as absorption spectra, emission (including blackbody or fluorescence) spectra, elastic scattering and reflection spectra, impedance (e.g., index of refraction) spectra, and/or inelastic scattering (e.g., Raman and Compton scattering) spectra, of analytes in the sample cell.

Non-optical techniques also can be employed. For example, with samples being reciprocated in and out of the reactor, in a sterile fashion, sample components can be analyzed using electrochemical sensors (for monitoring dissolved oxygen or other parameters), protein-based measurements, such as ELISA (enzyme-linked immunosorbent assay), flow cytometry, or other techniques currently known in the art or developed in the future.

Illustrative implementations described herein rely on near infrared (NIR) spectroscopy. Probing molecular overtone and combination vibrations, NIR spectroscopy covers the region of from 780 nanometer (nm) to 2500 nm wavelength of the electromagnetic spectrum. An overview of NIR spectroscopy can be found, for example, in an article by A.M.C. Davies in “An Introduction to Near Infrared (NIR) Spectroscopy”, http://www.impublications.com/content/introduction-near-infrared-nir-spectroscopy. See also, Cervera, A. E., Petersen, N., Lantz, A. E., Larsen, A. & Gernaey, K. V. Application of near-infrared spectroscopy for monitoring and control of cell culture and fermentation, Biotechnol. Prog. 25, 1561-1581 (2009); and Roggo Y, et al., “A review of near infrared spectroscopy and chemometrics in pharmaceutical technologies”, Journal of Pharmaceutical and Biomedical Analysis, Volume 44, Issue 3, 2007.

Among its strength, NIR spectroscopy presents a non-invasive, non-destructive investigative approach, typically involving fast scan times. A discussion of NIR as applied to microfluidic and other systems is provided in U.S. patent application Ser. No. 16/419,690, to Hassell et al., filed on May 22, 2019, published on Nov. 28, 2019 as U.S. Patent Application No. 2019/0358632A1, and incorporated herein in its entirety by this reference.

Samples to be analyzed, e.g., by NIR or another suitable method, are obtained using a sample tube that can be inserted into a vessel, e.g., a bioreactor or another type of vessel or arrangement used to conduct biochemical or chemical processes. Examples include cell growth protocols, fermentations, and so forth. Bioreactors monitored as described herein can feature a suitable design and can be characterized by a specific volume or dimensions, as known in the art or as developed in the future.

In one implementation, techniques described herein are practiced with a bioreactor that houses or is a cell culture system for the three-dimensional assembly, growth and differentiation of cells and/or tissues. The bioreactor can contain cells, culture media, nutrients, metabolites, enzymes, hormones, cytokines and so forth. With many processes conducted in bioreactors requiring or benefiting from the stringent control of parameters such as pH, levels of oxygen, nutrients, metabolites and/or other species, the sample tube for extracting a sample from a bioreactor can be combined or integrated with a sample cell and components configured for NIR interrogation and analysis. In specific embodiment, the sample cell is disposable. In other embodiments, the sample cell includes components that are disposable.

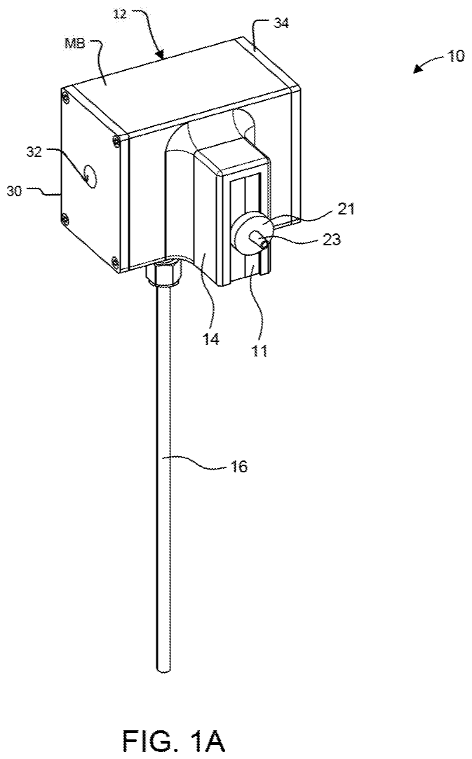

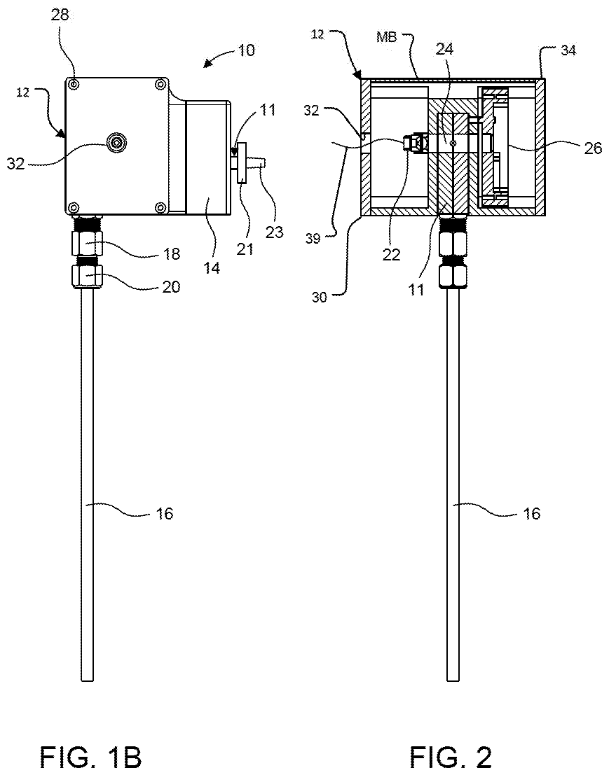

Shown in FIGS. 1A and 1B, for example, is device 10 including tether head 12, sample cell (also referred to as probe cell or flow cell) 11, typically disposable, and sample tube 16 that can be inserted in a bioreactor to extract and/or release a sample to be analyzed. To assemble the device, the sample cell 11 is mounted into the tether head 12. In the example of FIGS. 1A and 1B, sample cell 11 is inserted into access structure 14, where it can be locked in position using alignment screws or bolts or interference fit another suitable technique. One or more connectors (elements 18 and 20 in FIG. 1B) link sample tube 16 to tether head 12.

The cross-sectional view of FIG. 2 shows the optical elements and orientation of the sample cell 11 inside the tether head 12. As seen in this drawing, NIR radiation is introduced via optical cable 39. A collimator 22 such as a convex lens or gradient-index (GRIN) optics or lens, directs the light to intersect the sample cell 11 at scan area 24, which can be circular. Transmitted radiation is detected by NIR detector 26 on the opposite side of the sample cell. An arrangement in which NIR electromagnetic radiation is transmitted directly from the laser launch fiber 39, through the sample and to the detector 26 eliminates the need for other elements such as patch fiber optics, mirrors, etc., and thus optimizes the spectroscopic signal.

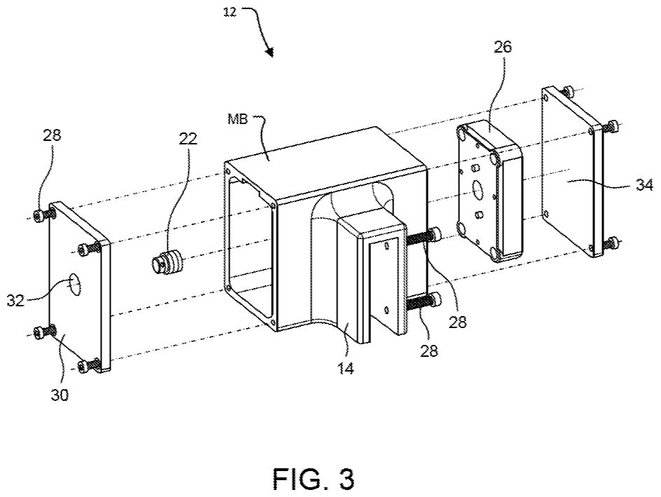

FIG. 3 is an exploded view showing how device 10, provided with aligning flow cell screws 28, for positioning a disposable sample cell, is integrated with a NIR arrangement that includes collimator 22 and detector 26. A first side cover 30 is provided with an opening 32 for the optical cable 39 transmitting incident NIR light. At the opposite end of tether head 12 is a second side cover 34 for sealing the detector 26 within the main body MB of the tether head 12.

More generally, device 10 can include components for conducting measurements using other types of electromagnetic radiation, such as, for instance, millimeter, microwave, terahertz, infrared (including near-, mid- and/or far-infrared), visible, and/or ultraviolet (UV). Further, the spectroscopic analysis employed can measure different characteristics of analytes in the sample. Examples include but are not limited to: absorption spectra, emission (including blackbody or fluorescence) spectra, elastic scattering and reflection spectra, impedance (e.g., index of refraction) spectra, and/or inelastic scattering (e.g., Raman and Compton scattering).

In some cases, device 10 is adapted for using non-spectroscopic methods to analyze constituents in a sample collected in sample cell 11 or to monitor sample parameters. For instance, the tether head 12 can be provided or integrated with one or more sensor(s) and/or other elements to measure pH, temperature, sample constituents (e.g., DO), and so forth. In one illustration, a protein in the sample is analyzed by a technique such as ELISA, and so forth.

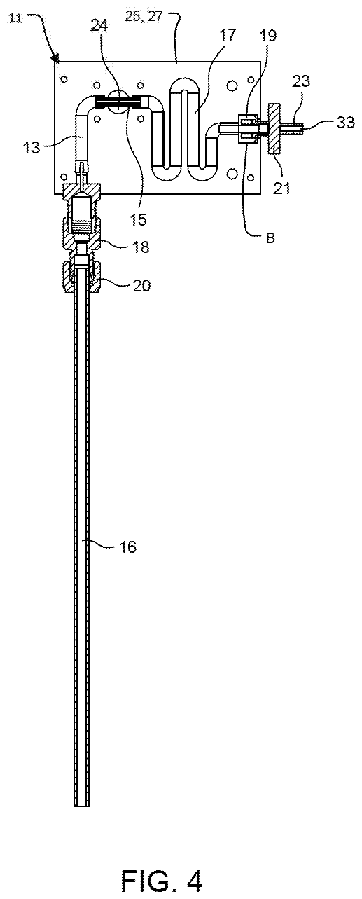

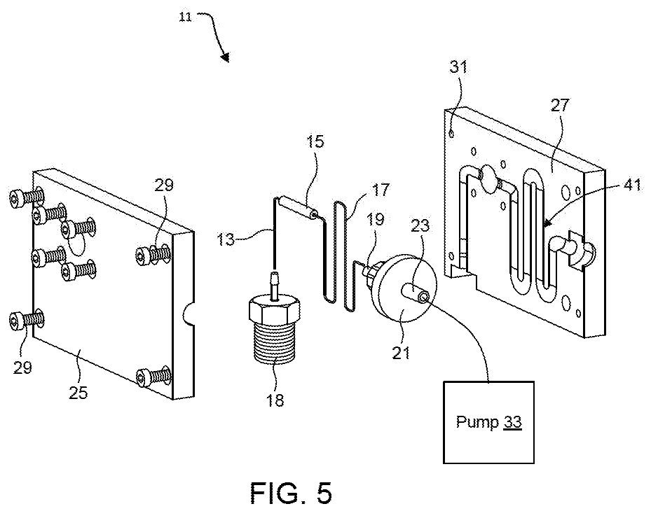

One implementation of sample cell 11, in relation to other components of the device 10, is shown in FIG. 4, while FIG. 5 is an exploded view of the sample cell 11. In the embodiment of FIGS. 4 and 5, sample cell 11 includes a first conduit, e.g., tubing channel 13, an interrogation cuvette 15 in fluid communication with the first conduit and a second conduit, e.g., tubing channel 17 in downstream communication with the curvette. The tubing channels 13, 17 are fabricated in two sandwiched plates 25 and 27. First tubing channel 13 is in fluid communication with sample tube 16 through connectors 18 and 20. In one example, connector 18 is a barbed fitting, while connector 20 is a compression fitting. Tube 16 and one or both connectors 18, 20 can be made of a material that withstands conditions in the reactor, is inert with respect to the contents of the reactor and does not introduce impurities to the process conducted in the bioreactor. Examples include stainless steel, thermoplastics (e.g., polypropylene or polystyrene), and others.

The distal downstream end of tubing channel 17 is configured for connection to a pump 33. One implementation uses connector 19 that is inserted and affixed in a bore B fabricated in the plates 25 and 27, a Luer lock fitting, for example. Beyond this connector is filter 21 and then a suitable conduit 23 for connecting to the pump 33.

In specific embodiments, filter 21 is a sterile filter, characterized by a pore size, that, in specific embodiments, is selected for reduced and preferably minimize resistance, thus allowing the pump to apply negative pressure, while preventing access of potential contaminants (viruses or bacteria) from the outside into the system, e.g., from conduit 23 into sample cell 11. Examples of suitable sterile filters include but are not limited to syringe-style filters commercially available from Sterlitech Corporation, Pall Corporation and other suppliers. Suitable sterile filters can be initially sterilized by gamma-ray radiation and rated for autoclave sterilization. In one implementation, the sterile filter has a pore size within the range of from about 0.22 to about 0.45 microns. It pore size is generally less than 100 microns.

In many embodiments, the pump 33 is a peristaltic pump, the peristaltic action of which can create a negative pressure. In contrast to traditional modes of operating peristaltic pumps, here, the peristaltic pump is utilized as a reversible/reciprocating pump, as further described below.

As illustrated in FIG. 5, the sample cell 11 comprises the two plates 25 and 27, which can be disposable (consumable). The two plates can be made from stainless steel, thermoplastics or another suitable material. Plates 25 and 27 are affixed to one another by means such as screws 29 aligned with corresponding holes 31, which can be threaded. Suitable adhesives, clamps, fasteners, etc. also can be employed to join together and/or align plates 25 and 27. In specific implementations, an inner face of one or both plates is patterned to form features 41 that define the tubing channels 13, 17 and hold the curvette 15, and are configured to support, nestle or enclose the flow cell (or portion thereof), thereby minimizing any gap between the plates upon assembly. In some cases, the patterned features can be designed to define (form) one or more segments of the sample cell.

In contrast to many existing arrangements that employ flow-cells, the device and techniques described herein protect the cells extracted from the bioreactor by preventing, reducing or minimizing their circulation through the pumping system.

In some implementations, tubing channel 17, extending from the cuvette 15 to the sterile filter 21, is configured to provide a tortuous path. As shown in FIGS. 4 and 5, this tortuous path can be obtained by U-shaped sections or other suitable designs that can provide added volume for the collection of larger amounts of fluid from the bioreactor. In addition to ensuring that a representative sample is obtained, increasing the pathway for the bioreactor sample also reduces or minimizes the likelihood of bioreactor fluid from coming into contact with the sterile filter. Since most pumps cannot overcome the pressure needed to pump through a wetted filter, arrangements in which the sterile filter remains clear of bioreactor medium maintain the pumping resistance at manageable levels, allowing the pumping action to continue unhampered.

For the in-situ collection and analysis of samples from the bioreactor, sample tube 16 is connected to sample cell 11 which, in many cases, is sterilizable and/or disposable. The resulting apparatus can then be autoclaved with the bioreactor. Once clean and ready for experiments, the tether head, which houses the optics, is placed on top of the flow cell and aligned with the sample cells with the help of suitable aligning pins or aligning screws 28 (see FIG. 3), for example.

To withdraw a fluid sample from the bioreactor, a tube such as tube 23 in FIGS. 4 and 5 is connected to the peristaltic pump 33 which draws a sample from the bioreactor into the sample cell via negative pressure. Since cells are typically not drawn into the pumping system, the tube 23 extending beyond the sterile filter does not need to be sterilized.

The negative pressure can be applied for a time interval that is sufficient to obtain an adequate sample volume. For a manual and/or an automated approach, this time interval can be selected based on routine experimentation, mathematical modeling, prior experience, and so forth.

Once the sample has been collected into the sample cell, the sample can be analyzed, by NIR spectrometry, for example. Some implementations provide a common processor for controlling both the peristaltic pump as well as the NIR sample analysis. In other embodiments, the peristaltic pump 33 is part of a system for NIR analysis. Some illustrative approaches for providing and analyzing samples using NIR are described, for instance, in U.S. patent application Ser. No. 16/419,690 (U.S. Patent Application Publication No. 2019/0358632A1), to Hassell et al., filed on May 22, 2019 and incorporated herein in its entirety by this reference.

After the analysis is completed, reversal of the peristaltic pump 33 returns the sample to the bioreactor.

In one example, the frequency of measurement is set to typically less than 30 minutes or less than 15 minutes, and often about every 5 minutes or less. To be gentle on cells, the pump 33 is run slowly, with the sample taking about 1 minute to be pulled into the sample cell. In many instances, scanning is repeated multiple (two or more) times for averaging and quality control, to ensure good signals, for instance. The pump is then reversed, pushing the entire sample back into the reactor, until the sample tube is completely purged. After a suitable time interval, 5 minutes, for example, a subsequent sample is pulled into the tube. In many implementations, the sampling is repeated with any desired frequency over any desired time period. For example, sampling is repeated (e.g., at a few minute-intervals) to monitor the entire reactor process (e.g., for a week, two weeks, three weeks or longer).

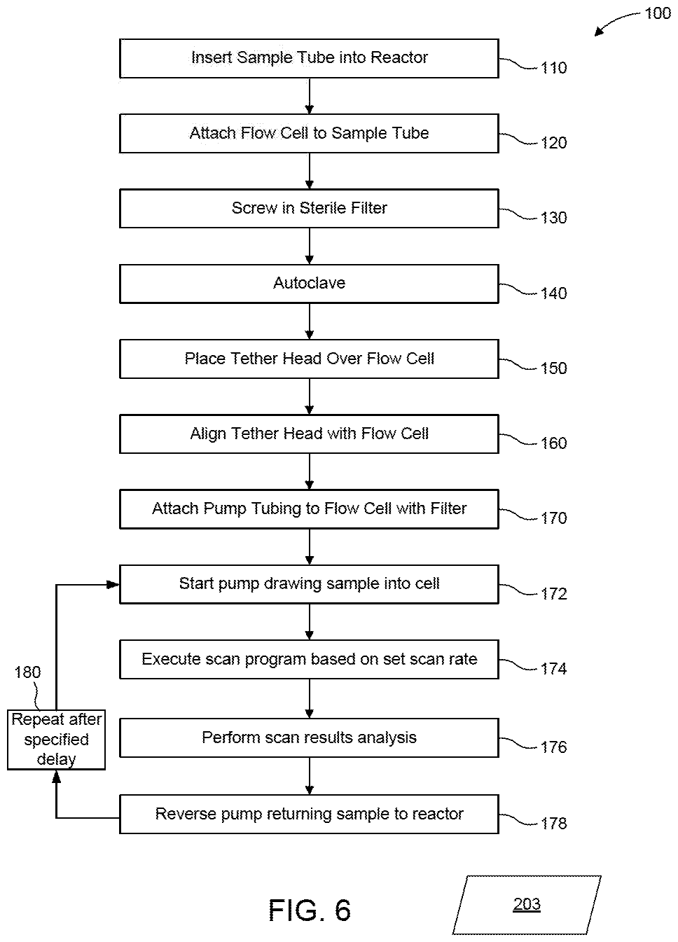

Details of a sample collection and analysis protocol 100 are shown in the flow chart of FIG. 6. In step 110 of the procedure, the sample tube (element 16 in FIGS. 1A and 1B) is inserted in a bioreactor. Step 120 involves attaching the flow cell (element 11 in FIGS. 4 and 5) to the sample tube, using, for instance, connectors 18 and 20 in FIG. 1A. In step 130, the sterile filter (element 21 in FIG. 4) is attached or mounted using, e.g., the Luer lock fitting 19 in FIG. 4. After autoclaving (step 140), during which the resulting assembly is sterilized, in an autoclave, for instance, the tether head (element 12 in FIGS. 1A and 1B) is placed over the sample cell 11 (step 150) and aligned with the flow cell (step 160). In step 170, a pump (e.g., pump 33) is connected to the filter end of the sample cell, using, e.g., tube 23 (see FIGS. 4 and 5) or other suitable conduits. The pump 33 is started under the control of a controller 203, drawing a sample into the cell (step 172). A desired spectral scan rate is set and the scanning program is executed in step 174 by the controller. Performing the analysis of the scan is illustrated by 176. In step 178 the pump is reversed, returning the sample to the reactor. Steps 172 through 178 can be repeated, e.g., after a specified delay, as shown by loop 180.

At least some of the steps in procedure 100 can be automated by using controller 203. For example, controller 203 can start and control the peristaltic pump to draw and maintain a sample in the flow cell. It can also reverse the operation of the pump to return the sample back to the reactor.

If a spectroscopic technique such as NIR is employed, controller 203 includes a light source that generates the optical signal on optical cable 39. The controller then monitors the response of the NIR detector 26, a photodiode, for example. In some implementations, the controller's light source is a narrow band tunable light source such as a tunable laser to interrogate specific wavelengths or wavelength bands of the electromagnetic spectrum to perform absorption spectroscopy on the sample in the sample cell. The controller 203 operates the laser to sweep through the spectral scan band at the desired spectral scan rate. The controller 203 can further include a single board computer, for monitoring the response of the photodiode as a function of the instantaneous wavelength of the tunable laser in order to resolve the absorption spectrum of a material in the sample. The controller 203 typically also includes the drive and control electronics for operating the pump 33.

Aspects of the invention can be practiced using other device configurations. An example is an arrangement in which the tortuous path described above is replaced by a straight pathway. Additionally, or in the alternative to this straight pathway, the device can be modified with respect to the geometry (orientation) of the tether head relative to the sampling tube that is inserted in the bioreactor, fittings used, means of support, and/or other construction details.

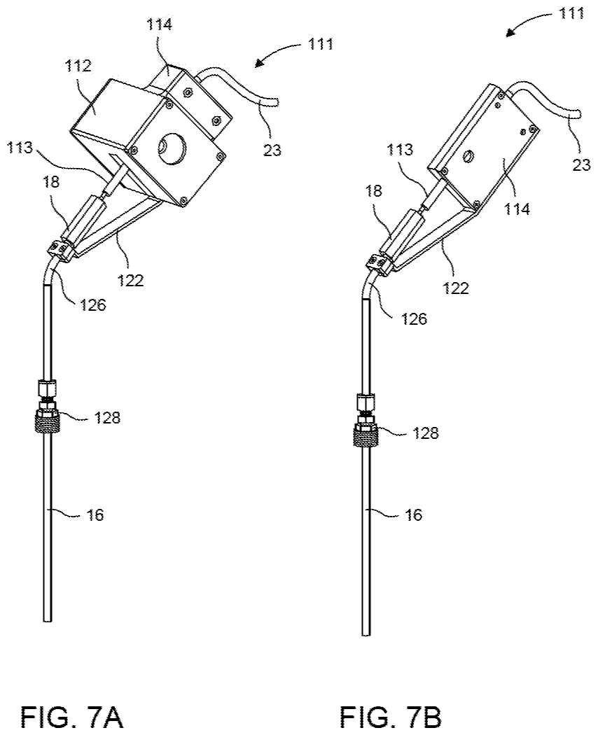

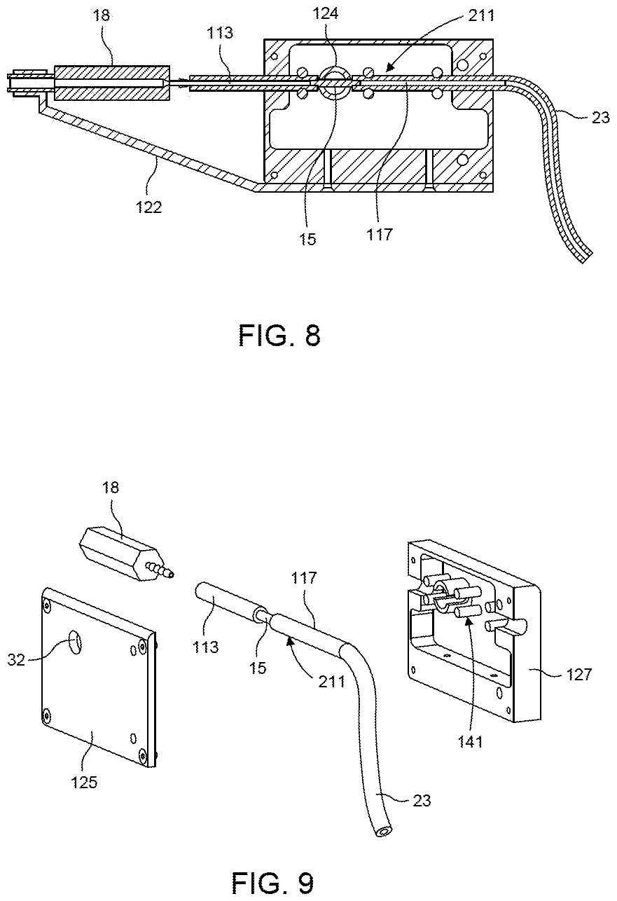

Shown in FIGS. 7A, 7B, 8 and 9, for example, is device 111, including sample tube 16 for collecting samples from a bioreactor, and tether head 112. The sample cell can be inserted in access structure 114 and includes a straight pathway defined by tubing channel 117, which extends from cuvette 15 to conduit (tubing) 23, used for connecting to a peristaltic pump.

As seen in FIG. 9, sample cell 211 is sandwiched between plates 125 and 127, typically disposable (consumable). The inner face of one or both plates can be patterned to form recessed and/or protruding features 141, configured, for instance, to define, support, nestle or enclose sample cell 111 (or portion thereof), thus minimizing any gap between the plates upon assembly. In some cases, the patterned features can be designed to define (form) one or more segments of the sample cell.

Sample tube 16 and tubing channel 113 (which leads to cuvette 15) are arranged to form a 45° angle (see, e.g., tubing section 126 in FIGS. 7A and 7B). Other suitable angles can be selected. The design can further incorporate means of support such as support bracket 122 and/or a height adjustable fitting, element 128. Cuvette 15 provides a (circular) scan area 124, where an optical path defined by optical elements housed in the tether head 112 intersects the sample cell. In specific implementations, the optical elements are essentially as described with reference to FIGS. 2 and 3.

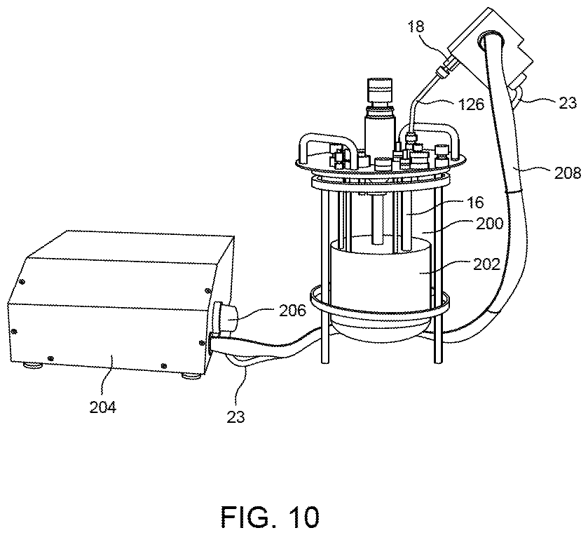

Monitoring a bioreactor with a device such as described herein is illustrated in FIG. 10. As shown in this figure, a device such as, device 111 of FIGS. 7A and 7B, having, for instance, tether head 112 and a sample tube 16, extending into a bioreactor 200, is used to sample and analyze the contents of the bioreactor, e.g., the reactor medium 202. One or more instruments 204 can provide a pumping system that includes peristaltic pump 206 (which connects to the sample cell via tubing 23), an analysis system, computer processing and/or other functions. Instrument 204 can include the controller 203, described above with reference to FIG. 6.

In some embodiments, a sample extracted from the bioreactor 200 via in-situ sample tube 16 and collected in a sample cell, such as described with reference to FIG. 4, 5, 8 or 9, for instance, is analyzed by NIR absorption spectrometry. NIR incident light can be transmitted from a tunable laser (housed, e.g., in instrument 204) to the tether head (see, e.g., element 112 in FIG. 7A) and signal from the detector within the tether head can be returned to instrument 204 using a wire harness 208, for example.

As already noted, other spectroscopic or non-spectroscopic approaches can be employed to analyze sample constituents and/or sample parameters. Whether the analysis employed relies on NIR spectroscopy, another spectroscopic or a non-spectroscopic method, the peristaltic pump is operated to collect the sample in the sample cell and reversed to return the sample to the bioreactor once the desired measurement has been completed.

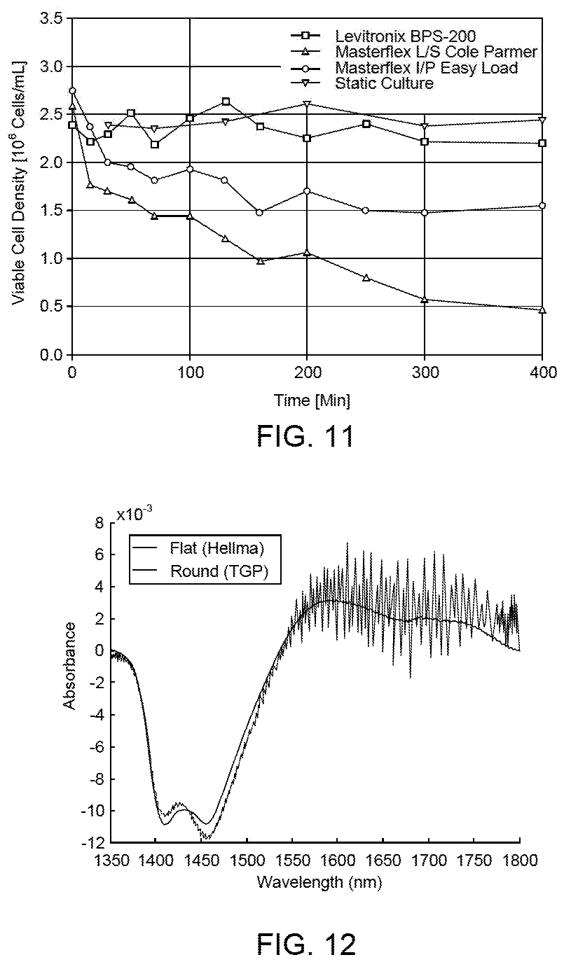

Advantages associated with arrangements that reduce, minimize or prevent cell handling (drawing the cells through the pumping system, for example) are illustrated in FIG. 11. The data show the impact of various techniques on viable cell densities. A levitating pump, for example, does not involve much cell touching and yields good cell viability. For cells that are not drawn and circulated in the pumping system, as described herein, results are expected to look very similar to those obtained with the static culture.

In some embodiments, the signal to noise ratio is greatly increased by replacing the common quartz cuvettes, having flat surfaces, with round (also referred to herein as cylindrical) quartz cuvettes. Such rounded sample probes can control reflections caused by parallel surfaces in the path of a light beam, as these reflections can interfere with one another and the incoming light, thus generating noise purely due to the positioning of the light source, the cuvette for monitoring a sample, and the detector. Another advantage of replacing the traditional square cuvettes with round ones is cost-related. Producing parallel quartz surfaces, such as found in the standard cuvette, yields a very expensive component (>$100), whereas round (cylindrical) quartz can be extruded in large lengths at a time, yielding relatively inexpensive cuvettes (e.g., less than $1).

FIG. 12 highlights the impact of the flat versus round surfaces for making measurements. The sample cells used in the comparison were a Hellma flat-wall cuvette and TGP (Technical Glass Products, Inc.) quartz tubing. The results indicate that a round cuvette can give a signal that is improved by an order of magnitude.

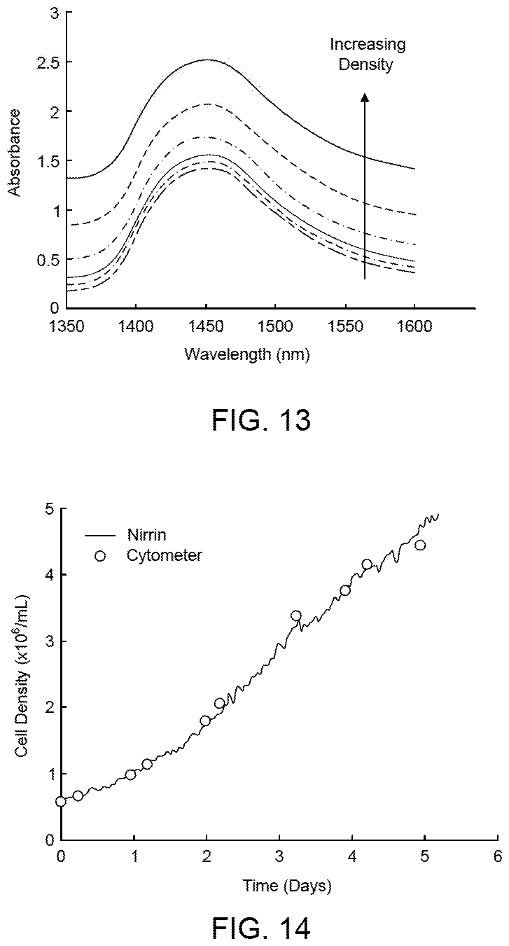

Techniques described herein can be applied in various situations. In one example, the process parameter monitored is cell growth. Shown in FIG. 13, for example, are scans of samples of increasing cell densities in the NIR wavelengths, leading to increased scatter of the beam and thus an apparent increased absorbance.

FIG. 14 compares the in-situ monitoring of CHO cells grown in a bioreactor. From spiking cells and then counting off-line, it is possible to build a calibration model, which then may be used to monitor the growth of cells in a more complex bioreactor.

In one example, embodiments of the invention are applied to the field of cell and gene therapy. Typically, such treatments involve collecting cells from a subject's body, modifying (or reprogramming) the cells and growing these cells to a number suitable for re-implantation.

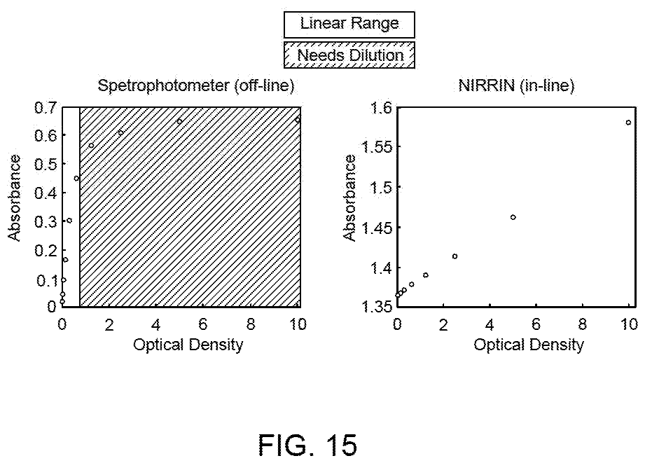

While cell and gene therapies are expected to expand rapidly in the coming years, a remaining key challenge for researchers and producers is assessing these complicated, living medicines during manufacturing. As developed by NIRRIN Bioprocess Analytics, Inc., Billerica, Mass., the use of NIR laser technology, which has the ability to precisely measure cell growth rates and quantify key metabolites in cell cultures, offers a highly useful mechanism for achieving this goal. Techniques that utilize the probe and method described above, coupled with NIR or other analytical approaches can be integrated into complex cell and gene therapy production processes, providing valuable insight into cellular behavior and phenotypes. An illustration is presented through FIG. 15, which compares samples of Pichia being grown in a shake flask and studied using NIR measurements, performed with an in-line probe taking samples automatically and reading absorbance at approximately 1450 nm, with off-line measurements from a standard spectrophotometer.

In a specific example, aspects of the invention can be applied to the production of chimeric antigen receptor T (CAR-T) cells. This process begins with the collection and purification of a patient's own lymphocytes, which are then genetically engineered to target specific cell surface markers and expanded to create a therapeutic infusion product. Analysis of cell growth and density is critical to this process, as the U.S. Food and Drug Administration (FDA) requires each CAR-T batch to contain a minimum number of cells. In addition, assessment of cellular phenotype via measurement of secreted metabolites, cytokines, and/or other factors can offer insight into the manufacturing process. By applying techniques described herein, this information can be obtained without the need for manual sampling, increasing efficiency and reducing the risk of contamination. In addition to CAR-T therapies, applications can also target the production of allogeneic CAR-T cells, tumor infiltrating lymphocyte therapies, induced pluripotent stem cell treatments, and other ex vivo cell or gene therapy product.

In sum, procedures and techniques relying on NIR laser technology have the potential to enter the cell and gene therapy production process and provide important insight into cell quality and therapy development. From determining cell number and density to precisely measuring secreted factors of interest (in real time, using a device and/or method such as described herein) there are a number of valuable uses for biomanufacturers working on next generation therapies.

While this invention has been particularly shown and described with references to preferred embodiments thereof, it will be understood by those skilled in the art that various changes in form and details may be made therein without departing from the scope of the invention encompassed by the appended claims.

What is claimed is:

1. A device for monitoring a bioreactor, the device comprising:
a sample cell including:
a first end connectable for fluid communication with a sample tube for collecting a sample from a bioreactor; and
a second end connectable for fluid communication with a pump,
wherein the sample cell is mountable onto a tether head which includes one or more optical elements for analyzing the sample.

2. The device of claim 1, wherein the sample cell is disposable.

3. The device of claim 1, further comprising a sterile filter at the second end.

4. The device of claim 1, wherein the sample cell includes a round cuvette.

5. The device of claim 1, wherein the sample cell includes a tortuous fluid path.

6. The device of claim 1, wherein the sample cell includes a straight fluid path.

7. The device of claim 1, wherein the pump is a peristaltic pump.

8. The device of claim 1, wherein the optical elements include elements for near infrared interrogation and/or detection of analytes.

9. The device of claim 1, wherein the optical elements form an optical path that intersects the sample cell at a scan area.

10. A method for monitoring a bioreactor process, the method comprising:
a. operating a pump to generate a negative pressure in a sample cell;
b. drawing medium from a bioreactor through a sample tube to collect a sample in the sample cell;
c. analyzing the sample; and
d. operating the pump to generate a positive pressure, thereby releasing the sample from the sample cell, through the sample tube, and into the bioreactor.

11. The method of claim 10, wherein the sample is analyzed by near infrared spectrometry.

12. The method of claim 10, wherein the sample is analyzed by a spectroscopic or a non-spectroscopic method.

13. The method of claim 10, wherein a. through d. are repeated.

14. The method of claim 10, wherein cells in the sample are not circulated through a pumping system.

15. The method of claim 10, wherein access of contaminants from a pumping system into the sample cell is prevented by a sterile filter.

16. The method of claim 10, further comprising assembling and/or disassembling the sample tube, the sample cell, the peristaltic pump and a tether head containing one or more elements for analyzing the sample.

17. The method of claim 10, further comprising autoclaving the sample tube and/or the sample cell.

18. A device for monitoring a bioreactor in-situ, the device comprising:
a sample tube for extracting a sample from a bioreactor;
a tether head housing one or more optical components;
a peristaltic pump;
a sample cell that is mountable onto or into the tether head, the sample cell having a first end that is connectable to the sample tube and a second end that is connectable to the peristaltic pump; and
a sterile filter at the second end.

19. The device of claim 18, wherein the sample cell includes a round cuvette.

20. The device of claim 18, wherein the sample cell includes a tortuous fluid path.

21. The device of claim 18, wherein the sample cell includes a straight fluid path.

22. The device of claim 18, wherein the optical components include components for NIR interrogation and/or detection of analytes.

23. A system comprising:
a bioreactor;
a probe that includes:
a sample tube immersible in the bioreactor;
a sample cell having a first end configured for fluid communication with the sample tube and a second end configured for fluid communication with a pump;
a sterile filter separating the sample cell from the pump;
a tether head containing elements for analyzing a sample in the sample cell and configured to cover the sample cell;
a controller for operating the pump, analyzing the sample, or both.

24. The system of claim 23, wherein the pump is operated as a reciprocal pump.

25. The system of claim 23, wherein the elements for analyzing the sample define an optical pathway that intersects the sample cell at a scan area.

26. The system of claim 23, wherein the elements for analyzing the sample are configured for NIR spectrometry.

27. The system of claim 23, wherein the bioreactor is or contains a cell culture system for the three-dimensional assembly, growth and differentiation of cells and/or tissues.

28. A device for monitoring a bioreactor, the device comprising:
a sample cell having:
a first end connectable for fluid communication with a sample tube for collecting a sample from a bioreactor;
a second end connectable for fluid communication with a peristaltic pump configured for operating as a reciprocal pump;
a sterile filter at the second end; and
a system for analyzing the sample.
